(19) United States
(12) Patent Application Publication
(10) Pub. No.: US 2007/0243578 A1
(43) Pub. Date: Oct. 18, 2007

(54) Method, system, apparatus and kit for semen analysis

(76) Inventors: Ken Mullen, Santa Fe, NM (US); Christina Noftsker, Santa Fe, NM (US)

Correspondence Address: KEVIN LYNN WILDENSTEIN, 9400 HOLLY AVENUE NE, BUILDING 4H, ALBUQUERQUE, NM, 87122, US

(21) Appl. No.: 11/787,150

(22) Filed: Apr. 13, 2007

Related U.S. Application Data
(60) Provisional application No. 60/792,922, filed on Apr. 18, 2006.

(51) Int. Cl. G01N 33/48

(52) U.S. Cl. 435/40.5

(57) ABSTRACT

The present invention is generally directed towards a method, system, apparatus and/or kit for collecting samples of bodily fluids, and more specifically towards collection of supplies that enable a lay person to collect and analyze a semen sample for use at home for male fertility or in the animal husbandry arts. In one embodiment, the semen analysis method includes a sperm count or determination of sperm concentration or sperm density, and may include an evaluation of motility and/or morphology. An exemplary kit may include at least a microscope, microscope slides, cover slips and other items may be included in the kit to facilitate the analysis method (such as, for example, instructions; microscope attachments; specialized microscope slides that create a fixed volume or depth, hemacytometer slides, counting chambers, or similar slides that have a fixed volume or depth and/or rulings to define an area thus defining a volume; specialized microscope slides that incorporate stains or other materials to aid in visualizing, analyzing, or counting sperm; pipettes, transfer pipettes, or disposable pipettes for transferring the sample from the collection container to the microscope slide; graduated cylinders or other devices for measuring volume; semen standard solutions; semen collection vessels and/or semen collection condoms) and any combination of the forgoing corresponding supplies.

### CLAIM OF PRIORITY

The present invention claims priority based upon U.S. Provisional Patent Application No. 60/792,922, filed Apr. 18, 2006, which is hereby incorporated by reference.

### FIELD OF INVENTION

The present invention is generally directed towards a method, system, apparatus and/or kit for collecting and analyzing samples of bodily fluids, and more specifically, towards a portable test kit which enables a lay person to collect and analyze a semen sample containing one or more sperm for use locally and real-time for male fertility or in the animal husbandry arts. The semen sample may be from any animal, including, but not limited to, horse or stallion, cow or bull, swine or boar, dog, cat or other animal, and is more particularly suited for humans. The test kit analysis is adapted to analyze, at least, any one or more of the following: sperm count, sperm density, sperm concentration, sperm motility, sperm morphology, or semen volume.

### BACKGROUND OF THE INVENTION

It is well known that more than 10% of couples experience difficulty in conceiving a pregnancy. The causes of infertility are typically divided into three categories: issues arising from the male species; issues arising from the female species; or issues arising between the couple or other unidentified issues. Generally, practitioners recommend that a couple try to conceive for at least six months to a year before any testing procedure is taken. If a couple has not conceived within the approximate one year time frame, tests may then be initiated to evaluate the fertility of both partners. A physician typically orders a semen analysis from the male first. This is a logical first step, since the test is invitro. However, one significant drawback is that the physician's visit and the semen analysis lab work is typically very expensive. In most cases, health insurance does not cover fertility testing or treatments.

Moreover, while many males may come to believe that a single semen test is sufficient to provide accurate results, a single semen analysis may (and typically, does) not provide an accurate picture of the available semen. Rather, sperm counts and motility can vary widely over time (motility describes how much the sperm are moving). If the male who provided the samples had only provided a few samples at the low points as seen in FIG. 1, he would have incorrectly concluded that he was sub-fertile.

Many things can affect sperm count and motility. A visit to a hot tub or a bodily fever can kill most if not all the sperm in, the male's body. The sperm life cycle in a human male is about 75 days, so it takes about two and one half months for sperm motility to return. Likewise, changes in diet or life style are not reflected in the semen analysis for several months. A test kit designed for at home use would allow a male to test his semen multiple times to be certain his assessment of fertility is accurate.

However, many human males are reluctant to initiate fertility testing because they are embarrassed by the process. For example, the sample must first be collected by the male. Then, the sample must be delivered to the lab warm and within one hour of collection. Because of these constraints, the sample is usually collected at the lab, rather than the privacy of one's residence. At most labs, this means the sample must be collected in the bathroom or other private area with the noise of people just outside the door. And, after the semen sample is generated, the patient must deliver it to the receptionist with other people standing around. Many men find the process embarrassing and unpleasant, and thus, resist following the process. In other cases, men believe they have no basis for having their fertility evaluated and hesitate to initiate the process.

Typically, no evaluation is performed until the couple has been trying to conceive for a year. Many researchers have suggested that male fertility is declining. Jensen et al. suggest that the declining birthrates in industrialized countries are due, in part, to decreased male fertility. Recent results from Denmark show that more than 30% of 19 year old men have sperm counts in the subfertile range. And, many couples try to conceive as the woman's fertility is declining with age. The delay increases the couples stress levels and may cause them to miss their best opportunity for conception. If a reliable, portable test kit and method were available, a couple could discover issues with the male's fertility and initiate corrective actions a year or more earlier than is typical with the current state of the art. A portable kit and method would minimize the embarrassment, expense, and delay in evaluating the male's fertility. Eliminating this delay may help couples to initiate professional fertility evaluation and treatment a year or more sooner than with the present state of the art.

The present invention allows couples to conveniently test the male's fertility on a regular or systematic basis. This type of testing is helpful in cases where fertility treatments have been undertaken. Some examples of such treatments include vasectomy reversals and surgical treatment for varicoceles.

The present invention is also applicable to the husbandry arts. For example, much of modern livestock reproduction is accomplished by collection of a semen sample followed by artificial insemination. The present invention provides a valuable adjunct to this process. With the present invention, one can evaluate collected semen prior to shipment, sale, or insemination. The present invention is also an aid to appropriately diluting a sample using semen extender. The present invention can also be used to evaluate purchased semen to determine, on a real-time basis, if the sperm counts and motility are as expected before using the semen for artificial insemination.

At least one calorimetric sperm concentration test kit is currently available in many drug stores. In this test kit, a color indicator is used to determine if sperm concentration is above or below the minimum fertility standard of 20 million/mL. The World Health Organization (WHO) suggests that less than 20 million sperm per milliliter is sub-fertile. However, this colorimetric test kit provides no information about sperm motility or sperm health. Since no quantitative information is available from this calorimetric test kit, a human male cannot track changes in sperm count.

To provide results indicative of sperm health and motility, another semen test kit exists which forces the sperm to swim through a barrier designed to mimic a woman's cervix. The Fertell male fertility test is an over the counter single use in vitro diagnostic test that screens for male fertility. The test measures the number of sperm that get through the barrier. A red line indicates a positive test. While this test does not provide quantitative information, it does indicate whether an adequate number of the sperm are sufficiently motile.

PCT Application WO 2006/000813 discloses a kit suitable for evaluating a semen sample, the kit having a microscope, sample support, sample locating means, cover slip, a graticule on the sample support or cover slip, an illuminating means, and a spacer for creating a predetermined sample depth. This disclosure is deficient for several reasons. For example, it requires a graticule formed immediately on the sample support or cover slip to thereby define an area and a spacer to maintain a predetermined depth to define a volume. As such, the overall cost of this type of a kit will necessarily lead to higher user costs for use of a specially designed sample support or cover slip. Moreover, those of skill in the art realize that the purpose of this disclosure's graticule can easily be accomplished by conventional microscopes (e.g., use of a reticule in the microscope (as seen for example in FIG. 5) to determine an area and a fixed cover slip slide to create a known depth to define the sample volume. The use of a reticule in the microscope is advantageous because only one ruling is required for any number of analyses, thus potentially lowering the overall cost of the system. Additionally, this disclosure is deficient because it discloses a sample locating means which prevents the user from moving the slide to view other areas of the slide, and further discloses the use of a proprietary slide whenever a sample is viewed. Consequently, it would be more advantageous to have a kit which would allow a user to move the slide to allow for counting and evaluating the sample in multiple places on a conventional, low cost, off-the-shelf microscope slide.

European Patent No. EP 0437408 describes a fixed cover slip microscope slide located in a temperature controlled holding device that is placed under a microscope to be used for counting sperm cells. However, this disclosure fails to recognize the use of a microscope slide in a kit for use by lay persons.

In view of the foregoing, there is a need for a portable semen test kit, method, apparatus or system adapted to provide a real-time visual test where lay users or couples can see the sperm, determine sperm count, and evaluate motility at any location. A visual test eliminates the abstract nature of color change tests and makes the analysis more insightful. The present invention includes at least one microscope and corresponding supplies that make it possible for lay users or couples to visually evaluate semen quality. The kit and method allow couples to perform and track multiple analyses over time so they can better understand changes in fertility.

### SUMMARY OF THE INVENTION

The following summary of the invention is provided to facilitate an understanding of some of the innovative features unique to the present invention, and is not intended to be a full description of variations that may be apparent to those of skill in the art. A full appreciation of the various aspects of the invention can be gained from the entire specification, claims, drawings, and abstract taken as a whole.

The present invention is generally directed towards a method, system, apparatus and/or kit for collecting samples of bodily fluids, and more specifically, a portable test kit which enables a lay person to collect and analyze a semen sample for male fertility or in the animal husbandry arts. In one embodiment, the kit comprises a pre-determined combination of one or more supply components which assists the non-professional in performing semen analysis. The kit components may include (but is not limited to): one or more microscopes; microscope attachments; microscope slides and/or cover slips; predefined microscope slides adapted to create a fixed volume or depth to facilitate accurate counting of sperm; hemacytometer slides, counting chambers, or similar slides that have a fixed volume or depth and/or rulings (such as, for example, viewable indicators) which define an area thus defining a volume; specialized microscope slides that incorporate stains or other materials to aid in visualizing or evaluating sperm; prepared slides of sperm or semen, stains or other materials that aid in visualizing, analyzing, or counting sperm; pipettes, transfer pipettes, or disposable pipettes; graduated cylinders or other devices for measuring volume; semen standard solutions consisting of particles that are similar in size to sperm or sperm heads; semen collection vessels; and/or semen collection condoms. Any combination of the foregoing supplies (whether singular or plurality) may be included in the method, system, apparatus or kit.

The semen analysis method may include a sperm count or determination of sperm concentration or sperm density, and may also include an evaluation of body fluid or sperm motility and/or morphology. An exemplary kit for use in this method may include at least a microscope, microscope slides, cover slips and other items may be included in the kit to facilitate the analysis method (such as, for example, instructions; microscope attachments; specialized microscope slides that create a fixed volume or depth, hemacytometer slides, counting chambers, or similar slides that have a fixed volume or depth and/or rulings to define an area thus defining a volume; specialized microscope slides that incorporate stains or other materials to aid in visualizing, analyzing, or counting sperm; pipettes, transfer pipettes, or disposable pipettes for transferring the sample from the collection container to the microscope slide; graduated cylinders or other devices for measuring volume; semen standard solutions; semen collection vessels and/or semen collection condoms) and any combination of the forgoing corresponding supplies.

The novel features of the present invention will become apparent to those of skill in the art upon examination of the following detailed description of the preferred embodiment or can be learned by practice of the present invention. It should be understood, however, that the detailed description of the preferred embodiment and the specific examples presented, while indicating certain embodiments of the present invention, are provided for illustration purposes only because various changes and modifications within the spirit and scope of the invention will become apparent to those of skill in the art from the detailed description, drawings and claims that follow.

### BRIEF DESCRIPTION OF THE DRAWINGS

The accompanying figures further illustrate the present invention and, together with the detailed description of the preferred embodiment, assists to explain the general principles according to the present invention.

FIG. 1 graphically illustrates sperm counts taken every other week over a two year time span based upon a healthy male subject (indicating the natural variation in sperm counts over time, according to the World Health Organization);

FIG. 2 illustrates an exemplary microscope system within the scope of the subject invention, including a compound microscope with objective lenses on a turret, where two objective lenses are visible;

FIG. 3 illustrates a representative sample volume which may be defined as the area times the depth of the sample;

FIG. 4 illustrates a side perspective view of a representative microscope illuminating the semen sample (in the microscope slide) from above; and

FIG. 5 illustrates an optical view of a representative reticule as seen through a microscope lens.

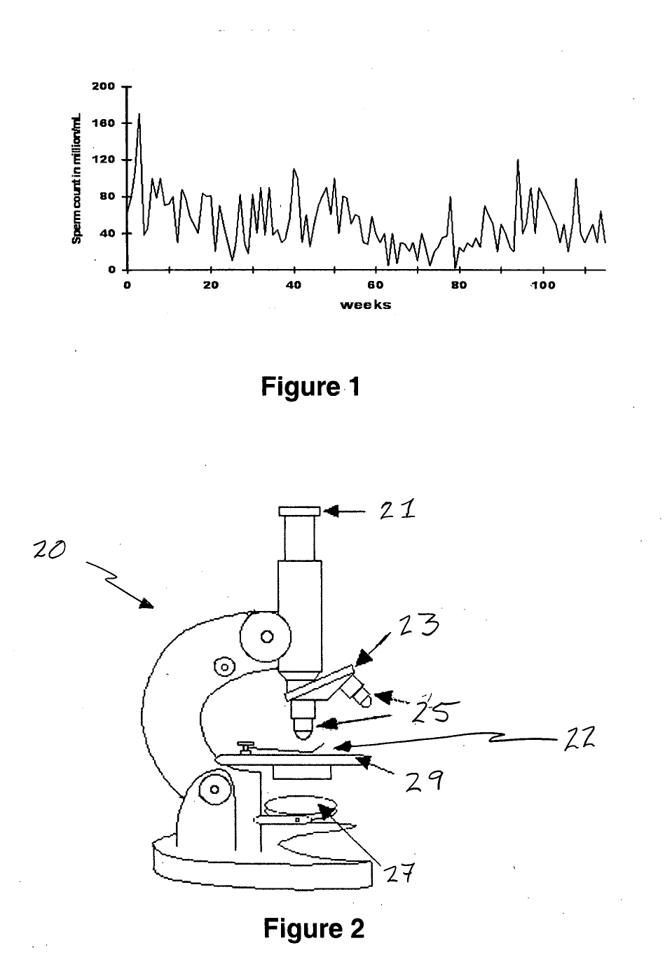

Additional aspects of the present invention will become evident upon reviewing the non-limiting embodiments described in the specification and the claims taken in conjunction with the accompanying figures, wherein like reference numerals denote like elements.

### DETAILED DESCRIPTION OF THE PREFERRED EMBODIMENT

The present invention is directed towards, in one embodiment, a kit adapted for collecting samples of bodily fluids, and more specifically, a kit allowing a user to collect and analyze one or more semen samples. The kit or component assembly is optimally used for performing portable, real-time semen analysis including determining sperm count, sperm density, sperm concentration, sperm motility and/or morphology of sperm.

In one preferred embodiment, an exemplary kit disclosed by the present invention includes at least one microscope 20 (such as shown in FIG. 2), and at least one user slide sample (shown as collective element 30 in FIG. 4) which may be adapted to retain bodily fluid for determining sperm count, sperm density, sperm concentration, sperm motility or morphology of sperm, or any combination thereof.

A representative portable microscope is shown in FIG. 2. Microscope 20 comprises an eyepiece lens 21 connected to at least one objective lens turret 23 and optically connected to at least one objective lens 25 having a primary objective lens 25a. Microscope 20 further includes a light source 27 at an opposite end from eyepiece lens 21, and a stage 29 which supports a representative microscope slide (not shown) and allows the user to position the slide, and sample on the slide, the stage 29 being in the same or approximately equivalent optical path as the eyepiece lens 21, the primary objective lens 25a and the light source 27. Preferably, stage 29 is adapted to retain the microscope slide 31 when the microscope 20 is agitated (as may occur, for example, out in the open field due to environmental conditions such as wind).

Those of skill in the art will realize that the term “microscope” also includes a digital microscope that may not include the foregoing conventional components of a microscope.

As graphically indicated in FIG. 2, the present invention supports using any portable microscope 20 with sufficient magnification and resolution to enable the user to optically view sperm cells. Sperm are generally viewed at 100 to 500 power. In a preferred embodiment of the invention, microscope 20 is a compound microscope. A compound microscope has more than one lens, 25 and 21. The objective lenses may be switchable using a lens turret 23. More than one objective lens allows the user to view the sample with different powers. Lower powers provide a larger field of view so more of the sample can be observed. In another embodiment, the microscope 20 may also be a simple microscope, having only one lens. In another embodiment the microscope 20 may also be a digital microscope having a camera connected to a computer (not shown), the digital microscope being adapted to replace or supplement the eyepiece 21. In this embodiment, the computer is optionally adapted to provide a corresponding image on an attached monitor. Moreover, the computer may be adapted to image the bodily fluid sample (such as semen) and assist a user to determine sperm count, sperm density, sperm concentration, sperm motility or morphology of sperm.

Those of skill in the art know that a microscope can be used to optically review the contents on a microscope slide covered with a cover slip, or can be used to review the contents without (e.g., uncovered) a cover slip. Preferably, microscope slides included in the present invention (illustrated as element 31 in FIG. 4) are adapted for use with a cover slip (illustrated as element 33 in FIG. 4) to retain the bodily fluid sample 35, to provide a uniform depth, and to prevent the objective lens from touching the sample and becoming soiled. As such, the present invention may include: a microscope slide 31 and cover slip 33; a microscope slide 31 having an attached fixed or removably attachable cover slip 33; a microscope slide 31 having predetermined materials thereon (such as stain, for example, or any like material which aids in visualizing, analyzing, or counting sperm); a microscope slide 31 with rulings or other visual indicators (not shown in FIG. 4); or a hemacytometer (not shown); any of these variations being hereafter defined as the “user slide sample” 30. Those of skill in the art will also recognize that a representative microscope slide may be, in one embodiment, substantially flat, without any cavity.

When the bodily fluid to be tested is obtained, the fluid can be applied to the user slide sample 30 through methods known in the art, such as through a disposable pipette, transfer pipette, or an eye dropper. The user slide sample 30 is adapted to receive and support the bodily fluid for optical review in a microscope. When the user slide sample 30 is filled with fluid and is ready for optical review, the user slide sample 30 is placed on the microscope's holding stage 22 such that the user slide sample 30 is in the optical path of the microscope 20.

In those embodiments where the user slide sample 30 includes a fixed cover slip 33, the bodily fluid may be distributed across the microscope slide 31 through capillary action. Namely, this process requires the user to place a small drop of the bodily fluid in a well at the edge of the microscope slide 31 through a pipette or like device. The fluid then flows under the cover slip 33 by capillary action. There are several manufacturers of slides with fixed cover slips that create a fixed and reproducible depth of sample.

As identified above, the user slide sample 30 may also include rulings or other visual indicators to assist the user in visually quantifying the sample 35. Many commercially available slides 31 (or, their corresponding cover slips) have rulings or other visual indicators which make it easy for the user to establish both the depth and the area of optical review.

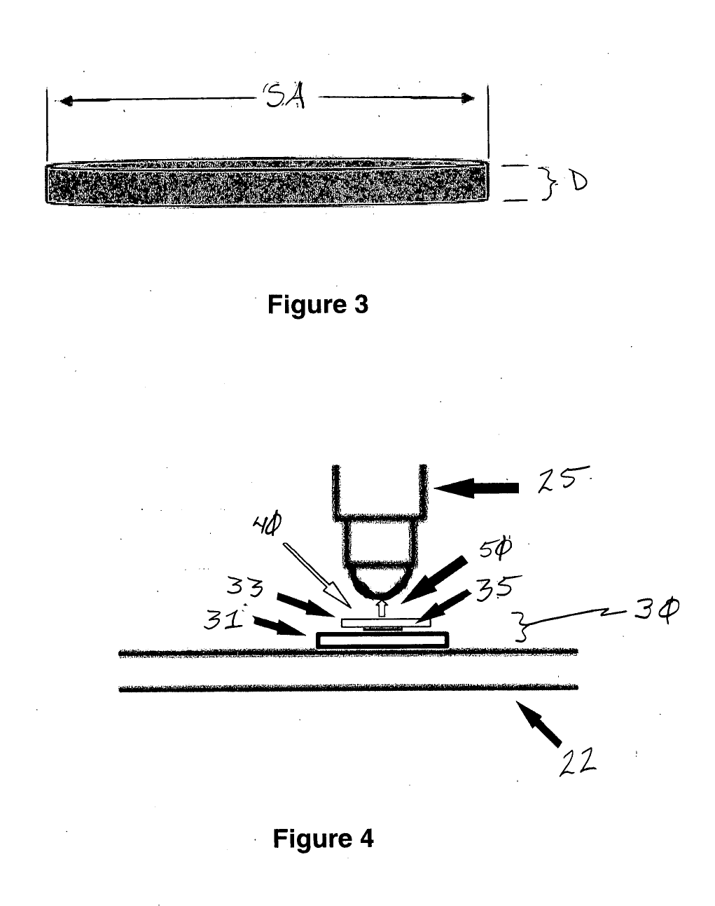

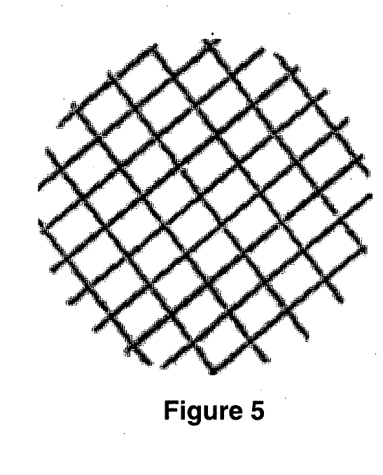

In other cases, the microscope's field of view (or, a reticule in the microscope as exemplified in FIG. 5) can be used with a fixed cover slip 33 to determine the area and depth of the sample. As seen in FIG. 3, the area of a collected sample on a microscope slide 31 is defined by the optical field of view of the microscope 20. With a fixed cover slip 33 attached to the microscope slide 31, the depth of the optical field of view is fixed. A volume can be determined by calculating the product of the surface area SA and the depth D of the field of view. Based upon the bodily fluid's volume in the sample 35, the user can calculate the approximate number of sperm to determine the number of sperm per unit volume. A conversion factor may then be applied to convert this value to millions of sperm per milliliter.

To determine sperm count or sperm concentration, the user counts the sperm in a defined volume. There are several ways to establish a volume. For example, if a microscope slide 31 with a fixed cover slip 33 is used, the depth is defined when the slide 31 is manufactured, and may typically include a cavity of between 4 and 20 microns deep. Because the sperm head is approximately 4 microns across, the cavity of the microscope slide 31 should be deep enough to allow the sperm to swim freely but not so deep that there are multiple layers of sperm in the optical field of view. The volume in which the number of sperm are counted is then determined by establishing an area using the microscope field of view as illustrated in FIG. 3, a reticule in the microscope as illustrated in FIG. 5, or rulings or other visual indicators formed on the slide 31 or cover slip 33. The volume is the product of the depth and the surface area. The instructions may give the user a conversion factor to allow the user to easily convert between the average number of sperm counted in a field of view to the standard units of million sperm per milliliter. In another embodiment of the present invention, a hemacytometer is used to establish the depth and the area viewed for sperm counting. To determine the total number of sperm in a sample, the sperm count (in million per milliliter) is multiplied by the total volume. The total volume of the sample may be measured with a graduated cylinder or other device for measuring volume.

As noted above, stains or other materials that aid in visualizing, analyzing, or evaluating the semen sample can also be added to the sample (or, an aliquot of the sample) in a liquid or dry form. As such, the kit of the present invention may also include a microscope slide 31 with a stain or like material already applied thereon. Use of a stain-applied microscope slide 31 is useful for evaluating morphology. In the case of a post vasectomy semen analysis, or, a very low sperm concentration, use of a stain or like material can be helpful in confirming that there are no sperm present. In this embodiment, the bodily fluid sample 35 is placed on the pre-stained slide and a cover slip 33 is placed over the sample 35. The slide 30 is then placed in the optical path of the microscope so the sample 35 may be evaluated.

It may be further helpful to provide a user with an optional clean or sterile sample collection container (not shown) as inclusive in the proposed kit. A clean sample collection container prevents contaminating the semen sample 35, because a contaminated semen sample will likely cause erroneous results because of soaps or other compounds present in the sample collection container. In another embodiment, the sample collection container could include a bodily fluid condom (such as a semen collection condom) adapted to collect bodily fluid such as a semen sample. Most conventional condoms are toxic to sperm and should not be used to collect a sample. However, Apex Medical Technologies, Inc. is one manufacturer of a biologically inert polyurethane condom that is designed to retain and maintain semen motility and velocity, thereby qualifying as a sample collection container.

An optional prepared comparative slide (not shown) may be included with the kit to visually assist an untrained user to learn to use or otherwise obtain a correct focus setting on the microscope. In this optional embodiment, a prepared comparative slide is a microscope slide already containing real or simulated stained sperm with the cover slip already adhered securely. The prepared comparative slide allows a user to first optically view the shape and size of sperm when viewed through the microscope 20 so that when a live semen sample is viewed, the user may then make a comparative optical review to ensure that the user is correctly using the microscope.

The foregoing disclosure describes a representative kit for evaluating a semen sample by use of a microscope. It is also possible to determine the sperm concentration in a semen sample directly by optical measurements. In particular, the concentration of sperm is proportional to the light scattered away from the path of the incident light. In many configurations, the light scattered off the sample 35 is measured at 90 degrees to the incident beam of light. This optical approach is typical for turbidity measurements made using nephelometry. The same technique can be applied to a semen sample, with the intensity of the scattered light being proportional to the concentration of sperm in a semen sample. Similarly, optical absorbance can be used to determine the sperm concentration in a sample, because the amount of light absorbed by the sample is proportional to the concentration of sperm in a sample.

Some in the art have suggested that dark field illumination enhances the image of sperm under a microscope. The foundation for this suggestion is that scattered light from the sperm does not get washed out by light passing through the sample. However, the present invention contemplates preferably illuminating the sample (from above the user slide sample) to improve image quality. Namely, as seen in FIG. 4, the user slide sample is illuminated from a point above the user slide sample. This means of illumination from a point above the user slide sample is preferable because the majority of light collected by the objective lens is scattered or reflected off of bodily fluid particles in the sample (such as sperm), thereby allowing ease of optically reviewing the sample for comparison purposes. In particular, the illuminating light beam (shown as element 40 in FIG. 4) is directed at the sample from above such that the reflected beam is not captured by the objective lens. Thus, most of the light that is captured by the objective lens 25a is scattered or otherwise reflected light (shown as element 50 in FIG. 4) from particles (such as sperm) in the sample.

Some users may have difficulty focusing the microscope. Sometimes, an inexperienced user visually focuses on the cover slip when using the microscope 20 instead of focusing on the sample 35 under the cover slip 33. In order to enable an untrained user to make test slides and practice counting and determining a concentration of particles before analyzing a semen sample, a semen standard solution may optionally be included in the kit. A semen standard solution is a solution which contains beads or particles that are similar in size to sperm or sperm heads (or, otherwise mimics the size and shape of sperm). The beads or particles can be viewed through microscope 20. An exemplary semen standard solution is adapted to allow a user to perform a complete test count using the semen standard solution. In operation, the semen standard solution is placed in the user slide sample 30 and placed in the microscope 20 for viewing by the user. The solution allows a user to view and count the beads or particles to confirm the user's ability to focus the microscope and count particles. The semen standard solution allows an inexperienced user to practice optically adjusting the microscope 20 before collecting a bodily fluid sample. The semen standard solution is treated exactly like a sample of bodily fluids. If the concentration of the particles in the semen standard solution is known, the user can then optically perform a test count through the microscope 20 to determine whether the approximate correct count may be achieved. A standard semen solution also allows the user to perform a complete count from beginning to end with a sample of known concentration.

The semen standard solution may comprise representative beads, spheres, or like particles. The semen standard solution may include latex beads (or microspheres) of approximately four (4) microns in diameter. This diameter is approximately the size of a typical human sperm head, so the user is provided an experience very similar to using a real semen sample. Other exemplary beads or particles can be made of latex, polystyrene, silica or other materials. In another embodiment, beads or particles may be adapted to attach to sperm to facilitate sperm counting and/or evaluating motility. Beads, especially colored beads, may be easier to optically view than sperm by use of a microscope. In this embodiment, the user would count the beads attached to the sperm instead of the sperm. This embodiment could be accomplished with a lesser quality microscope since the beads are easier to optically view through such a microscope. In another embodiment, different colors of beads could be used. If one color of beads were added at a different concentration (for example, one tenth the concentration), the user could count the beads of lesser concentration and multiply by ten to determine the concentration of sperm corresponding to a particular color of beads.

In operation, a method for real-time collection and analysis of bodily fluid samples is also disclosed wherein a portable test kit is introduced, the kit comprising at least one microscope, and at least one user slide sample adapted to determine sperm count, sperm density, sperm concentration, sperm motility or morphology of sperm, or any combination thereof. The user slide sample is adapted to receive and retain at least one semen sample. Next, the kit instructs the user to first obtain a bodily fluid sample, place the bodily fluid sample on the microscope slide, and count the sperm in one or more locations on the microscope slide, calculate an average of the sperm counts, and multiply the average sperm count by a predetermined conversion factor to determine the user's sperm count. The slide should preferably be movable, thereby allowing a user to count sperm in several locations on the slide. In one embodiment, the microscope is adapted to provide a means of illuminating the sample from a point above the user slide sample. Optionally, as identified above, one or more optional components may be introduced in this method, including a semen standard solution, a stain-applied microscope slide, a sterile sample collection container or a prepared comparative slide.

For sperm to do their job, they need to be good swimmers Sperm count alone is not the only factor used to determine fertility. Good sperm counts without motility is unlikely to lead to pregnancy. Sperm motility can be segregated into four classes: 1) Rapidly progressive—swimming rapidly, generally maintaining a consistent direction; 2) Slowly progressive—moving slowly with some forward progression; 3) Non-progressive—thrashing about, but not going anywhere (or going in circles); or 4) Immotile—not moving.

Preferably, as part of the method disclosed herein, the kit will include instructions to allow a user to evaluate motility. Typically, a total of 200 sperm are evaluated to determine the representative motility in the sample. The area viewed through the microscope 20 is referred to as the field of view. In general, each field of view is evaluated twice through the microscope. In total, this means evaluating 400 sperm. First, the kit will allow the user to count rapidly and slowly progressive sperm. Then, the kit will allow the user to count the non-progressive and immotile sperm. The kit will then instruct the user to calculate an average percentage of sperm for each class. As a general rule, it is desirable to have more than 50% progressive sperm.

The present invention may be embodied as a method, system, kit, an apparatus or a device. Other variations and modifications of the present invention will be apparent to those of ordinary skill in the art, and is not limited except by the appended claims. The particular values and configurations discussed above may be varied, and are cited to illustrate particular embodiments of the present invention. It is contemplated that the use of the present invention can involve components having different characteristics as long as the principles disclosed herein are followed.

It should also be appreciated that the particular implementations disclosed herein are illustrative of the invention and its best mode, and are not intended to otherwise limit the scope of the present invention in any way. Benefits, other advantages, and solutions to problems have been described above with regard to specific embodiments. However, the benefits, advantages, solutions to problems, and any element(s) that may cause any benefit, advantage, or solution to occur or become more pronounced are not to be construed as critical, required, or essential features or elements of any or all the claims. As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus. Further, no element described herein is required for the practice of the invention unless expressly described as “essential” or “critical”.

1. A portable kit for collecting and evaluating bodily fluids, the kit comprising:
a) at least one microscope comprising a light source, an eyepiece lens optically connected to at least one objective lens and a stage, the eyepiece lens, objective lens, stage and light source all being within an approximately equivalent optical path; and
b) at least one microscope slide, each microscope slide adapted to retain bodily fluid thereon for determining sperm count, sperm density, sperm concentration, sperm motility or morphology of sperm, the stage adapted to hold the microscope slide so that the microscope slide is in the optical path when the microscope is agitated.

2. The portable kit of claim 1, the microscope slide further comprising a removably attachable cover slip above the microscope slide, each removably attachable cover slip adapted to retain the bodily fluid on the microscope slide.

3. The portable kit of claim 1, the microscope slide further comprising a secondary material thereon, the secondary material adapted to aid in the visualization, analysis or counting of bodily fluid on the microscope slide while viewed through the optical path.

4. The portable kit of claim 3, the secondary material comprising a stain.

5. The portable kit of claim 1, the microscope further comprising at least one reticule having one or more visual indicators to aid in the visualization, analysis or counting of bodily fluid on the microscope slide while the microscope slide is viewed through the optical path.

6. The portable kit of claim 1, the kit further comprising a sample collection container adapted to collect bodily fluid.

7. The test kit of claim 6, the sample collection container being a collection condom.

8. The test kit of claim 1 wherein the bodily fluid is semen containing one or more sperm.

9. The test kit of claim 1, the kit further comprising a prepared comparative slide and a pre-adhered cover slip, each comparative slide adapted to visually assist help an untrained user to obtain a correct focus setting on the microscope.

10. The test kit of claim 1, each prepared comparative slide containing real or simulated stained sperm.

11. The test kit of claim 1, the microscope's light source being positioned to illuminate the microscope slide at a point above the microscope slide.

12. The test kit of claim 8, the kit further comprising a semen standard solution containing particles adapted to mimic the size and shape of sperm.

13. The test kit of claim 12, the particles being beads, spheres, or like shaped particles approximating the size of a sperm head.

14. The test kit of claim 8, the kit further comprising particles, beads, spheres or like particles adapted to attach to sperm to visually assist an untrained user in performing sperm counting and/or evaluating motility.

15. A method for collecting and evaluating bodily fluids, the steps comprising:
a) introducing at least one microscope, the microscope comprising a light source, an eyepiece lens optically connected to at least one objective lens, the eyepiece lens, objective lens and light source all being within an approximately equivalent optical path;
b) introducing at least one microscope slide from within this, each microscope slide adapted to retain bodily fluid on one surface of the microscope slide for determining sperm count, sperm density, sperm concentration, sperm motility or morphology of sperm;
c) instructing a user of the method to first obtain a semen sample containing sperm, place the semen sample on the microscope slide, and insert the microscope slide on the microscope stage; and
d) instructing and allowing a user of the method to count the sperm in one or more locations on the microscope slide, calculate an average of the sperm counts, and multiply the average sperm count by a predetermined conversion factor to determine the user's sperm count, sperm density, sperm concentration, sperm motility or morphology of sperm.

16. The method of claim 15, the microscope slide further comprising the steps of allowing a user of the method to count rapidly and slowly progressive sperm on the microscope slide, count non-progressive and immotile sperm on the microscope slide, and calculate an average percentage of sperm for each class of sperm motility.

17. The method of claim 15, each microscope slide further comprising one or more visual indicators placed thereon adapted to aid in the visualization, analysis or counting of bodily fluid on the microscope slide while viewed through the optical path.

18. The method of claim 15, the microscope's light source being positioned at a point above the microscope slide.

19. The method of claim 15 further comprising the steps of introducing a secondary material adapted to aid in the visualization, analysis or counting of bodily fluid, and mixing the secondary material with a portion of the semen sample prior to allowing a user to view the microscope slide through the optical path

20. The product according to the process of claim 15.

21. A portable kit for collecting and evaluating semen samples containing one or more sperm, the kit comprising:
a) at least one microscope comprising a light source, an eyepiece lens optically connected to at least one objective lens and a stage, the eyepiece lens, objective lens, stage and light source all being within an approximately equivalent optical path, the microscope's light source being positioned to illuminate the microscope slide at a point above the microscope slide;
b) at least one microscope slide, each microscope slide adapted to retain each semen sample on the microscope slide for determining sperm count, sperm density, sperm concentration, sperm motility or morphology of sperm, the stage adapted to hold the microscope slide so that the microscope slide is in the optical path, the microscope slide further comprising a secondary material thereon, the secondary material adapted to aid in the visualization, analysis or counting of sperm on the microscope slide while viewed through the optical path;
c) at least one sample collection container adapted to collect the semen sample;
d) at least one prepared comparative slide having a pre-adhered cover slip, each comparative slide adapted to visually assist help an untrained user to obtain a correct focus setting on the microscope; and
e) at least one semen standard solution containing particles adapted to mimic the shape of sperm when viewed through the microscope.

22. An apparatus for collecting and evaluating a semen sample containing one or more sperm, the apparatus comprising:
a) a digital microscope in electronic communication with a computer, the digital microscope having an optical path; and
b) at least one microscope slide, each microscope slide adapted to retain at least one semen sample thereon, the computer adapted to image the semen sample when placed in the optical path and assist a user in determining sperm count, sperm density, sperm concentration, sperm motility or morphology of sperm.
